(19) United States
(12) Patent Application Publication
(10) Pub. No.: US 2012/0296313 A1
(43) Pub. Date: Nov. 22, 2012

(54) Drug Coated Balloon Hemostatic Valve Insertion/Balloon Sheath

(75) Inventors: Anthony S. Andreacchi, San Jose, CA (US); Daniel L. Cox, Palo Alto, CA (US)

(73) Assignee: Abbott Cardiovascular Systems Inc., Santa Clara, CA (US)

(21) Appl. No.: 13/112,791

(22) Filed: May 20, 2011

(51) Int. Cl. A61M 25/10

(52) U.S. Cl. 604/509; 604/171; 604/103.02

(57) ABSTRACT

Introducer sheath for use in inserting a balloon catheter into a patient's vasculature includes a tubular member having an inner diameter, an outer diameter, a proximal end, a distal end, and a length therebetween, the inner diameter being sized to receive a catheter shaft having an attached expandable member in a deflated condition. The inner diameter can be varied from a first inner diameter at the proximal end and a second inner diameter at the distal end, the first inner diameter being greater than the second inner diameter. A method of inserting a balloon catheter into the vasculature of a patient is also disclosed. Additionally, a balloon catheter kit including a catheter and an introducer sheath is disclosed.

### BACKGROUND OF THE DISCLOSED SUBJECT MATTER

1. Field of the Disclosed Subject Matter

The disclosed subject matter relates to a device to aid the insertion of a catheter into the guide sheath and a method for using the same. Specifically, the disclosed subject matter relates to an insertion aid for catheters having an expandable member, such as a balloon or stent, positioned at a distal portion of the catheter, where the expandable member is coated with a therapeutic agent.

2. Description of the Related Art

Atherosclerosis is a syndrome affecting arterial blood vessels. It is characterized by a chronic inflammatory response in the walls of arteries, which is in large part due to the accumulation of lipid, macrophages, foam cells and the formation of plaque in the arterial wall. Atherosclerosis is commonly referred to as hardening of the arteries, although the pathophysiology of the disease manifests itself with several different types of lesions ranging from fibrotic to lipid laden to calcific. Angioplasty is a vascular interventional technique involving mechanically widening an obstructed blood vessel, typically caused by atherosclerosis.

During angioplasty, a catheter having a folded balloon is inserted into the vasculature of the patient and is passed to the narrowed location of the blood vessel at which point the balloon is inflated to the desired size by fluid pressure. Percutaneous coronary intervention (PCI), commonly known as coronary angioplasty, is a therapeutic procedure to treat the stenotic regions in the coronary arteries of the heart, often found in coronary heart disease. In contrast, peripheral angioplasty, commonly known as percutaneous transluminal angioplasty (PTA), generally refers to the use of mechanical widening of blood vessels other than the coronary arteries. PTA is most commonly used to treat narrowing of the leg arteries, especially, the iliac, external iliac, superficial femoral and popliteal arteries. PTA can also treat narrowing of carotid and renal arteries, veins, and other blood vessels.

Although the blood vessel is often successfully widened by angioplasty, sometimes the treated region of the blood vessel undergoes vasospasm, or abrupt closure after balloon inflation or dilatation, causing the blood vessel to collapse after the balloon is deflated or shortly thereafter. One solution to such collapse is stenting the blood vessel to prevent collapse. Dissection, or perforation, of the blood vessel is another complication of angioplasty that can be improved by stenting. A stent is a device, typically a metal tube or scaffold that is inserted into the blood vessel after, or concurrently with angioplasty, to hold the blood vessel open.

While the advent of stents eliminated many of the complications of abrupt vessel closure after angioplasty procedures, within about six months of stenting a re-narrowing of the blood vessel can form, a condition known as restenosis. Restenosis was discovered to be a response to the injury of the angioplasty procedure and is characterized by a growth of smooth muscle cells and extracellular matrix—analogous to a scar forming over an injury. To address this condition, drug eluting stents were developed to reduce the reoccurrence of blood vessel narrowing after stent implantation. A drug eluting stent is a stent that has been coated with a drug, often in a polymeric carrier, that is known to interfere with the process of re-narrowing of the blood vessel (restenosis). Examples of various known drug eluting stents are disclosed in U.S. Pat. Nos. 5,649,977; 5,464,650; 5,591,227, 7,378,105; 7,445,792; 7,335,227, all of which are hereby incorporated by reference in their entirety. However, drug eluting stents are not without limitations.

Drug coated balloons are believed to be a viable alternative to drug eluting stents in the treatment of atherosclerotic lesions. In a study which evaluated restenosis, and the rate of major adverse cardiac events such as heart attack, bypass, repeat stenosis, or death in patients treated with drug coated balloons and drug eluting stents, the patients treated with drug coated balloons experienced only 3.7% restenosis and 4.8% MACE (material adverse coronary events) as compared to patients treated with drug eluting stents, in which restenosis was 20.8 percent and 22.0 percent MACE rate. (See, PEPCAD II study, Rotenburg, Germany).

A drug coated balloon is a unique drug-device combination product. In addition to performing a dilatation function, the balloon delivers a therapeutic level of drug to the vascular tissue during an inflation that can last only a few seconds to several minutes. This rapid transfer of drug requires a coating capable of releasing a suitable amount of drug during the balloon inflation. There are a variety of potential mechanisms of drug transfer for a drug coated balloon, including: transfer of coating to the vessel wall with subsequent diffusion of drug into tissue; insertion of coating into tissues or fissures in the vessel wall produced by the dilatation; pressing the coating against the vessel wall, the drug dissolving into a thin liquid film to create a drug saturated boundary layer, and this dissolved drug diffusing into the tissue; and drug dissolving the entire time the balloon is near, or expanded against, the vessel wall and this dissolved drug diffusing into the tissue.

However, drug coated balloons present certain unique challenges. For example, the drug carried by the balloon needs to remain on the balloon during delivery to the lesion site, and released from the balloon surface to the blood vessel wall when the balloon is expanded inside the blood vessel. For coronary procedures, the balloon is typically inflated for less than one minute, typically about thirty seconds. The balloon inflation time can be longer for a peripheral procedure, however typically even for peripheral procedures the balloon is expanded for less than five minutes. Due to the short duration of contact between the drug coated balloon surface and the blood vessel wall, the balloon coating must exhibit efficient therapeutic agent transfer and/or efficient drug release during inflation. Thus, there are challenges specific to drug delivery via a drug coated or drug eluting balloon that are not present with a drug eluting stent.

Furthermore, conventional guide catheters and methods can pose problems when used in combination with drug coated balloon catheters. For example, the therapeutic agent can be wiped off the surface of the balloon during initial placement through the hemostatic valve of a conventional guide catheter, as well as during delivery through the tortuous lumen system.

In light of the foregoing, there is a need for an improved device to aid insertion of a balloon catheter having a therapeutic coating into the vasculature of a patient with minimal loss of the therapeutic agent. Thus, it is an object of the presently disclosed subject matter to provide a device that is useful for inserting a catheter having an expandable member with a therapeutic coating into the vasculature of a patient. It is also an object of the disclosed subject matter to provide a method for using the insertion aid.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes an introducer sheath. The introducer sheath includes a tubular member having an inner diameter and an outer diameter. The tubular member further has a proximal end and a distal end with a length defined therebetween. The inner diameter of the tubular member is sized to receive a catheter shaft having an attached expandable member in a deflated condition. The inner diameter can be varied from a first inner diameter at the proximal end and a second inner diameter at the distal end, with the first inner diameter being greater than the second inner diameter.

The introducer sheath can also include a flared portion having a proximal end and a distal end with the proximal end of the flared portion having an enlarged cross section and the distal end of the flared portion having a cross section substantially similar to the cross section of the proximal end of the tubular member. The introducer sheath can also include a breakable area of weakness along at least a portion of its length from the proximal to the distal end. For purposes of illustration and not limitation, the breakable area of weakness can be a perforation, a cut, or a slit. This perforation can be on one side of the sheath (allowing the sheath to be removed and opened like a ‘butterfly’, or on both sides (allowing the sheath to be removed and opened by a ‘peeling’ action). The introducer sheath further includes an inner surface and an outer surface and can have a lubricious coating applied to at least one of these surfaces. The introducer sheath can be made of any suitable material including but not limited to PEEK, HDPE, LDPE, FEP, PP, Kyner, or PTFE.

Further in accordance with another aspect of the disclosed subject matter, a balloon catheter kit is provided including a catheter and an introducer sheath. The catheter includes a tubular shaft having a proximal end a distal end and a length therebetween, and also includes an expandable member attached at the distal end of the tubular shaft. The introducer sheath includes a tubular member having an inner diameter and an outer diameter. The tubular member further has a proximal end and a distal end with a length defined therebetween. The inner diameter of the tubular member is sized to receive a catheter shaft having an attached expandable member in a deflated condition. The inner diameter can be varied from a first inner diameter at the proximal end and a second inner diameter at the distal end, with the first inner diameter being greater than the second inner diameter.

The introducer sheath can be sized such that its length completely encloses the length of the expandable member when inserted into the introducer sheath. The kit can be preassembled such that the introducer sheath is disposed over the expandable member of the catheter. The introducer sheath can also include a flared portion having a proximal end and a distal end with the proximal end of the flared portion having an enlarged cross section and the distal end of the flared portion having a cross section substantially similar to the cross section of the proximal end of the tubular member. The introducer sheath can also include a breakable area of weakness along at least a portion of its length from the proximal to the distal end. For purposes of illustration and not limitation, the breakable area of weakness can be a perforation, a cut, or a slit. This perforation can be on one side of the sheath (allowing the sheath to be removed and opened like a ‘butterfly’, or on both sides (allowing the sheath to be removed and opened by a ‘peeling’ action). The introducer sheath further includes an inner surface and an outer surface and can have a lubricious coating applied to at least one of these surfaces. The introducer sheath can be made of any suitable material including but not limited to PEEK, HDPE, LDPE, FEP, PP, Kyner, or PTFE.

The expandable member of the kit can have an inner surface and an outer surface and can further have a therapeutic coating disposed on its outer surface.

In accordance with another aspect of the disclosed subject matter, a method of inserting a balloon catheter into the vasculature of a patient is disclosed. The method includes providing a catheter, a guide catheter (or guide sheath), and an introducer sheath. The catheter includes a shaft with a proximal end, a distal end, and a length therebetween. The catheter further includes an expandable member attached at its distal end. The guide catheter includes a tubular member with a lumen defined therein. The introducer sheath includes a tubular member having an inner diameter and an outer diameter. The tubular member further has a proximal end and a distal end with a length defined therebetween. The inner diameter of the tubular member is sized to receive a catheter shaft having an attached expandable member in a deflated condition. The inner diameter can be varied from a first inner diameter at the proximal end and a second inner diameter at the distal end, with the first inner diameter being greater than the second inner diameter. The method further includes positioning the introducer sheath in a proximal end of the guide catheter and extending the expandable member through the introducer sheath and into the guide catheter.

The introducer sheath can also include a flared portion having a proximal end and a distal end with the proximal end of the flared portion having an enlarged cross section and the distal end of the flared portion having a cross section substantially similar to the cross section of the proximal end of the tubular member. The guide catheter can also include a hemostatic valve in which case positioning the introducer sheath includes inserting the introducer sheath through the hemostatic valve. The introducer sheath can also include a breakable area of weakness along at least a portion of its length from the proximal to the distal end. For purposes of illustration and not limitation, the breakable area of weakness can be a perforation, a cut, or a slit. This perforation can be on one side of the sheath (allowing the sheath to be removed and opened like a ‘butterfly’, or on both sides (allowing the sheath to be removed and opened by a ‘peeling’ action). The introducer sheath further includes an inner surface and an outer surface and can have a lubricious coating applied to at least one of these surfaces. The introducer sheath can be made of any suitable material including but not limited to PEEK, HDPE, LDPE, FEP, PP, Kyner, or PTFE.

Further, the method of insertion can include inserting the introducer sheath into the hemostatic valve before extending the expandable member into the introducer sheath, or alternatively, the expandable member can be extended into the introducer sheath before the introducer sheath is inserted into the hemostatic valve. The method can also include advancing the expandable member beyond a distal end of the guide catheter, and retracting the introducer sheath from the hemostatic valve.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the devices of the disclosed subject matter. Together with the description, the drawings serve to explain the principles of the disclosed subject matter.

### BRIEF DESCRIPTION OF THE DRAWINGS

FIG. 1 is a schematic side view of a representative introducer sheath in accordance with the disclosed subject matter.

FIG. 2 is a schematic perspective view of a representative introducer sheath showing a balloon catheter and hemostatic valve in accordance with the disclosed subject matter.

FIG. 3 is a schematic perspective view of the introducer sheath of FIG. 2 shown inserted through a hemostatic valve in accordance with the disclosed subject matter.

FIG. 4 is a schematic perspective view of the introducer sheath of FIG. 2 shown with a balloon catheter inserted into the introducer sheath in accordance with the disclosed subject matter.

FIG. 5 is a schematic perspective view of the introducer sheath of FIG. 2 shown with the distal end of the balloon catheter advanced past the distal end of the introducer sheath in accordance with the disclosed subject matter.

FIG. 6 is a schematic perspective view of the introducer sheath of FIG. 2 shown with the introducer sheath retracted from the hemostatic valve in accordance with the disclosed subject matter.

FIG. 7 is a schematic perspective view of the introducer sheath of FIG. 2 shown with the introducer sheath split into two pieces and removed from the catheter in accordance with the disclosed subject matter.

FIG. 8 is a schematic side view of a representative balloon catheter kit in accordance with the disclosed subject matter.

FIG. 9 is a schematic side view of the balloon catheter kit of FIG. 8 showing the balloon inserted into the introducer sheath and the introducer sheath inserted into the hemostatic valve in accordance with the disclosed subject matter.

FIG. 10 is a schematic side view of the balloon catheter kit of FIG. 8 showing the balloon advanced distally out of the introducer sheath in accordance with the disclosed subject matter.

FIG. 11 is a schematic side view of the balloon catheter kit of FIG. 8 showing the introducer sheath retracted proximally out of the hemostatic valve in accordance with the disclosed subject matter.

FIG. 12 is an end view of a representative guide sheath having an integral hemostatic valve in accordance with the disclosed subject matter.

FIG. 13 is a schematic view of a representative introducer sheath in accordance with the disclosed subject matter showing the introducer sheath intact, open on one side in a butterfly configuration, and peeled apart.

### DETAILED DESCRIPTION OF THE PREFERRED EMBODIMENT

Reference will now be made in detail to the preferred embodiments of the disclosed subject matter, an example of which is illustrated in the accompanying drawings. The disclosed subject matter will be described in conjunction with the detailed description of the system.

As disclosed herein, the devices and methods presented herein can be used for treating a variety of the luminal system of a patient. For purpose of illustration and not limitation, reference will be made to cardiovascular systems and blood vessels. In particular, the disclosed subject matter is particularly suited for enabling the insertion of a catheter having an attached expandable member, such as a balloon or stent, coated with a therapeutic agent into the cardiovascular system of a patient, such as for the performance of angioplasty and delivery of a therapeutic agent to a vasculature. The disclosed subject matter enables the insertion of the catheter while minimizing any loss of therapeutic agent or damage to the therapeutic agent coating of the expandable member.

In accordance with the disclosed subject matter, an introducer sheath is provided. The introducer sheath includes a tubular member having an inner diameter and an outer diameter. The tubular member further has a proximal end and a distal end with a length defined therebetween. The inner diameter of the tubular member is sized to receive a catheter shaft having an attached expandable member in a deflated condition. The inner diameter can be varied from a first inner diameter at the proximal end and a second inner diameter at the distal end, with the first inner diameter being greater than the second inner diameter. Additionally or alternatively, the introducer sheath can include a flared portion having a proximal end and a distal end with the proximal end of the flared portion having an enlarged cross section and the distal end of the flared portion having a cross section substantially similar to the cross section of the proximal end of the tubular member. The introducer sheath can also include a lubricious coating applied to either or both of its inner or outer surfaces. Further, the introducer sheath can include a breakable area of weakness along at least a portion of its length to facilitate removal of the introducer sheath from the catheter shaft or guidewire.

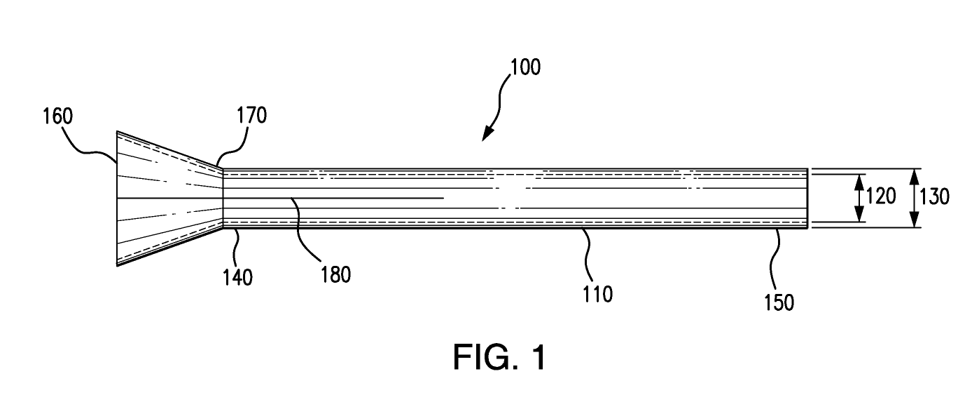

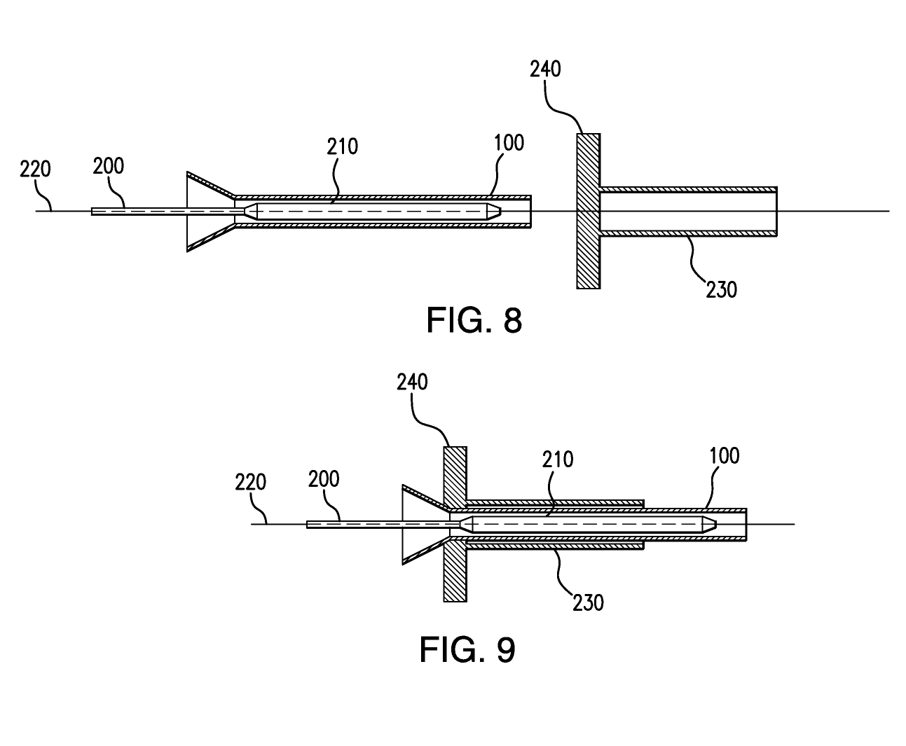

For purpose of explanation and illustration, and not limitation, an exemplary embodiment of an introducer sheath is shown schematically in FIGS. 1 and 8. Particularly and as illustrated, the introducer sheath 100 includes a tubular member 110 having an inner diameter 120 and an outer diameter 130. The tubular member further has a proximal end 140 and a distal end 150 with a length defined therebetween. The inner diameter 120 of the tubular member 110 is sized to receive catheter shaft 200 having an attached expandable member 210 in a deflated condition. The inner diameter can be constant along the length of the tubular member 110, as shown, or can be varied such as taper or the like from a first inner diameter at the proximal end and a second inner diameter at the distal end, the first inner diameter being greater than the second inner diameter.

The tubular member 110 of introducer sheath 100 can also include an enlarged or flared portion 160, 170. The flared portion includes a proximal end 160 and a distal end 170. The proximal end 160 of the flared portion has an enlarged cross section relative to its distal end 170, with the cross section of the distal end 170 being substantially similar to the cross section of the proximal end 140 of the tubular member. The wall thickness of the flared portion can be generally constant as shown, or can be varied such that the change in inner dimensions differs from the change in outer dimensions.

Additionally, introducer sheath 100 can have one or more breakable areas of weakness 180 along at least a portion of the length of the introducer sheath from the proximal end to the distal end. A breakable area of weakness can facilitate removal of the introducer sheath from the catheter shaft 200 or guidewire 220 by, for example, enabling ease of tearing of the introducer sheath. The breakable area of weakness can take on many forms, for example, a slit, a cut which extends partially through the wall thickness of the introducer sheath, perforations, or slots.

The inner and/or outer surfaces of the introducer sheath can be further coated with any of a variety of materials and techniques to enhance performance if desired, including a number suitable coatings and coating techniques subject to patent matters owned by Abbott Laboratories such as U.S. Pat. No. 6,541,116, U.S. Pat. No. 6,287,285, and U.S. Patent Publication No. 2002/0009535, the entireties of which are hereby incorporated by reference. For example, possible coating materials include lubricious materials such as Teflon® available from DuPont De Nemours, Wilmington, Del., U.S., and hydrophobic materials such as silicone lubricant dispersion PN 4097, available from Applied Silicone Corp., Ventura, Calif., U.S., or hydrophilic materials such as hydrogel available from Hydromer, Branchburg, N.J., U.S., or lubricious coatings such as those available from Hydro-Silk of Merritt Island, Fla., U.S. For example, a lubricious coating applied to the outer surface of the introducer sheath can reduce friction between the introducer sheath and the hemostatic valve when inserting the introducer sheath into the hemostatic valve and guide sheath. A lubricious coating applied to the inner surface of the introducer sheath can reduce the friction between the inner surface of the introducer sheath and the expandable member and catheter shaft enabling the expandable member and catheter shaft to be advanced more easily through the introducer sheath.

The introducer sheath can be formed of any suitable material. The introducer sheath can be single piece construction or an assembly of components. Suitable materials for the introducer sheath include, but are not limited to polymer materials such as nylon, urethane, polyurethane, polycarbonate, PEEK, PTFE, PVDF, Kyner, FEP, PP, PE, HDPE, or a multilayered constructions including L25, Plexar, or polyethylene of various suitable densities. As a further exemplary alternative, the introducer sheath can be constructed of a composite comprising a fabrication of several different materials, such as a co-extrusion of different polymers. For example and not limitation, exemplary embodiments can include a braided tube with a PTFE liner, a Polyamide middle layer with braiding and a Pebax 72D outer layer.

The tubular portion of the introducer sheath can have any suitable cross sectional shape, including elliptical, polygon, or prismatic, although a circular cross section or one that is similar to the cross section of the catheter shaft that will pass through the introducer sheath is generally preferred. Further, the introducer sheath can be of any suitable length. Preferably the length of the introducer sheath is greater than the thickness of the hemostatic valve through which it will have to pass. In a preferred embodiment the length of the introducer sheath is greater than the length of the expandable member of the catheter with which the introducer sheath will be used so that the expandable member can be fully encompassed by the introducer sheath. Such a configuration could also be used to protect the expandable member prior to use, for example, during packaging, sterilization, and shipping.

The introducer sheath can be manufactured using a variety of known techniques such as but not limited to: extrusion, blow molding, injection molding, air-blowing, stretching, deep drawing, polymerization, cross-linking, dipping from solution, powder depositioning, sintering, electro-spinning, melt spinning, deformation under temperature, stretch blowing, as well as classical machining technologies like milling, drilling, grinding, etc. Additionally, the introducer sheath can be constructed by an extrusion process using an extruder such as that available any of a number of known suppliers, such as Medical Extrusion Technologies, Inc. Murieta, Calif. U.S. Biosynthetic polymer materials can be constructed in a bioreactor according to the process disclosed in U.S. Pat. No. 6,495,152, the entirety of which is hereby incorporated by reference. The materials can be post processed in a number of ways including, for example and not by way of limitation, extrusion, molding, such as by injection or dipping, textile processing such as weaving or braiding, and forming. Forming processes that can be suitable are rolling and welding sheets of material or vacuum forming into tubular shapes, to name only a few examples.

The therapeutic agent can be applied by spraying, dipping, syringe coating, electrospinning, electrostatic coating, direct coating, roll coating, or a combination thereof. The therapeutic agent can be selected from the group consisting of zotarolimus, everolimus, rapamycin, biolimus, myolimus, novolimus, sirolimus, deforolimus, temsirolimus, paclitaxel, protaxel, or a combination thereof although other therapeutic agents are contemplated, including but not limited to the steroids dexamethasone, dexamethasone acetate, clobetisol, etc. The therapeutic agent can further comprise at least one compound selected from the group consisting of excipients, binding agents, plasticizers, solvents, surfactants, additives, chelators, or fillers. The excipient can be selected from the group consisting of contrast agents such as Ultravist® (lopromide) which is produced by Bayer Healthcare, polysaccharides, amino acids, proteins, non-ionic hydrophilic polymers, ionic hydrophilic polymers, acrylates, hydrophobic polymers, aliphatic polyesters and polyester block copolymers, and mucoadhesives. In one embodiment, the excipient is polyvinylpyrrolidone (PVP). In one embodiment, the plasticizer is glycerol.

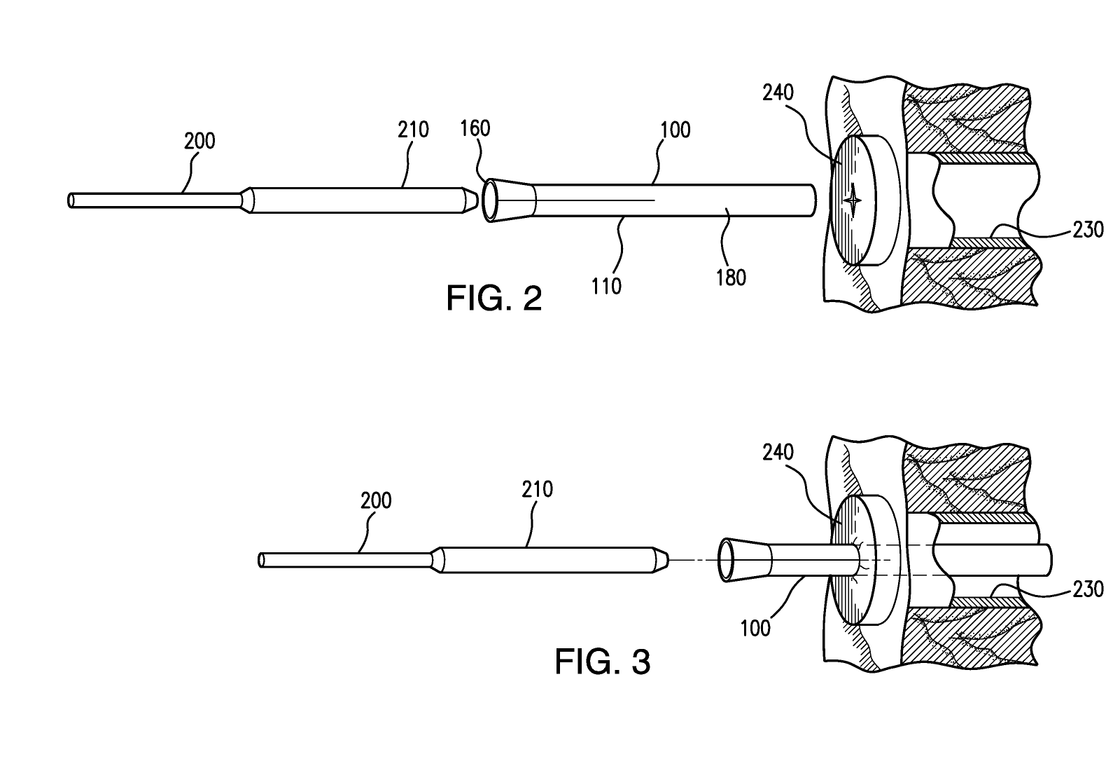

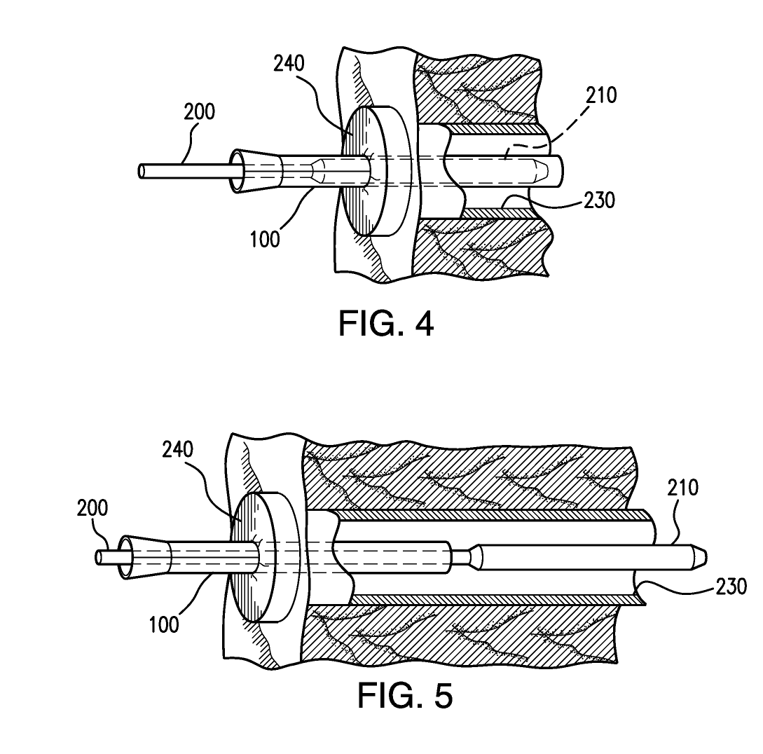

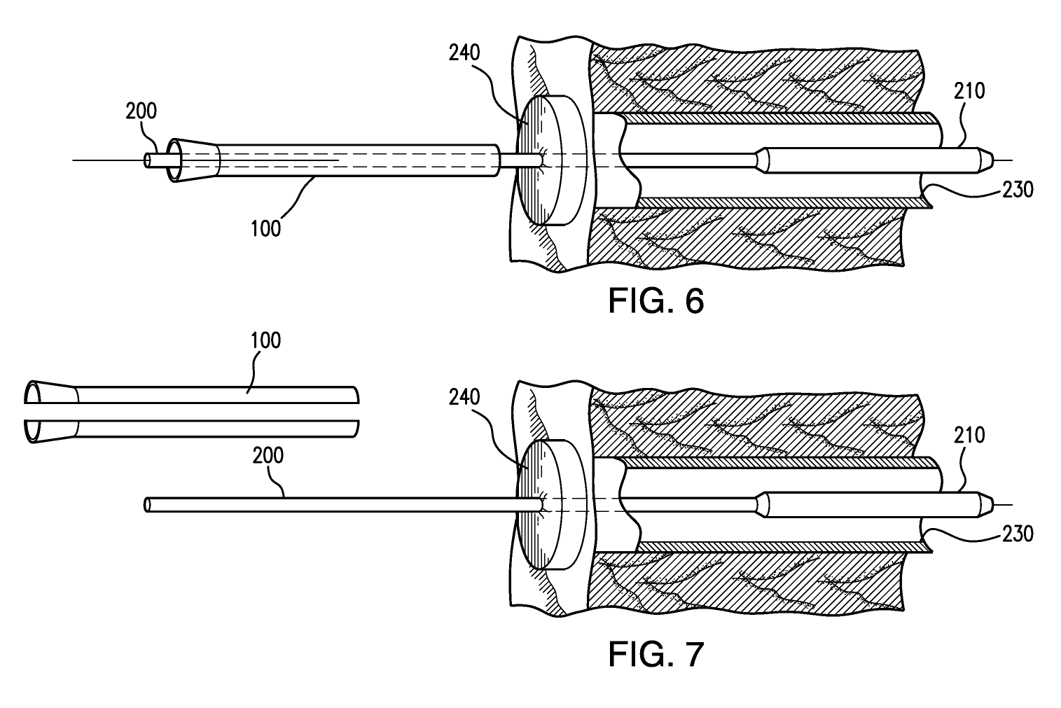

In accordance with another aspect of the disclosed subject matter the introducer sheath as described herein above can be used and/or provided in combination with a catheter. For purpose of illustration and not limitation, FIGS. 2 through 8 show schematic views depicting one method of using the introducer sheath in combination with a catheter in accordance with the disclosed subject matter. As shown in FIG. 2, guide catheter 230 including hemostatic valve 240, if desired as described further below, is positioned into the patient's vessel. FIG. 3 shows the introducer sheath 100 positioned through hemostatic valve 240 and into the guide catheter 230. As shown in FIG. 4, the expandable member 210 of balloon catheter 200 is positioned into introducer sheath 100. The distal end of the balloon catheter 200 is then advanced distally through introducer sheath 100 and beyond the distal end of the guide catheter into the patient's vasculature as shown in FIG. 5. Introducer sheath 100 is then removed from guide catheter 230 enabling hemostatic valve 240 to close as shown in FIG. 6. The introducer sheath can now remain on the catheter shaft. Alternatively, as described above, the introducer sheath can include one or more breakable areas of weakness and the method can include tearing or breaking the introducer sheath along areas of weakness to remove the introducer sheath from the catheter shaft and/or guidewire, as shown in FIG. 7.

For purpose of illustration and not limitation reference is made to FIGS. 8 and 9 showing schematic side views of a representative balloon catheter kit in accordance with another aspect of the disclosed subject matter. The catheter includes a tubular shaft 200 with a proximal end, a distal end and a length therebetween. The catheter further includes an expandable member 210 at the distal end of the tubular shaft, the expandable member having an inner surface, an outer surface and a length as known in the art. The catheter can be an over-the-wire configuration, or a rapid exchange configuration as further known in the art. Representative balloon catheters are described in more detail in U.S. Pat. Nos. 7,322,959, 6,383,212 and 6,206,852, which are incorporated in their entirety herein. Additionally or alternatively the expandable member can have a therapeutic agent applied to its outer surface. As embodied herein, the introducer sheath of the kit is disposed over the expandable member.

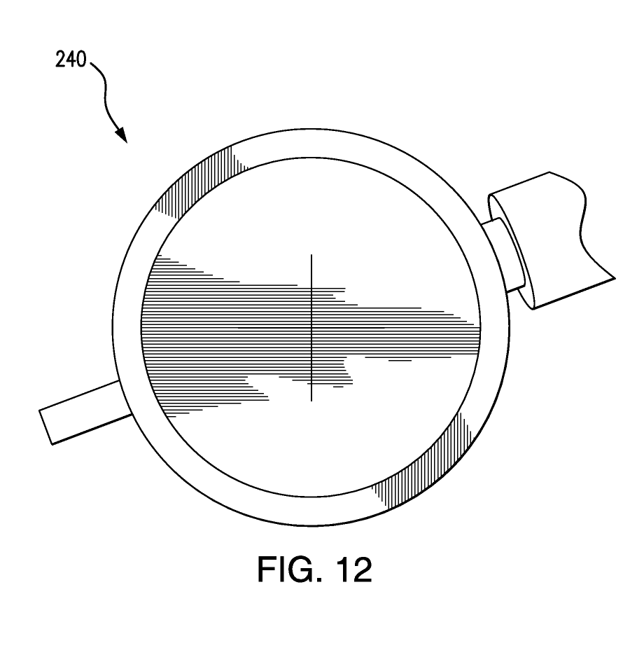

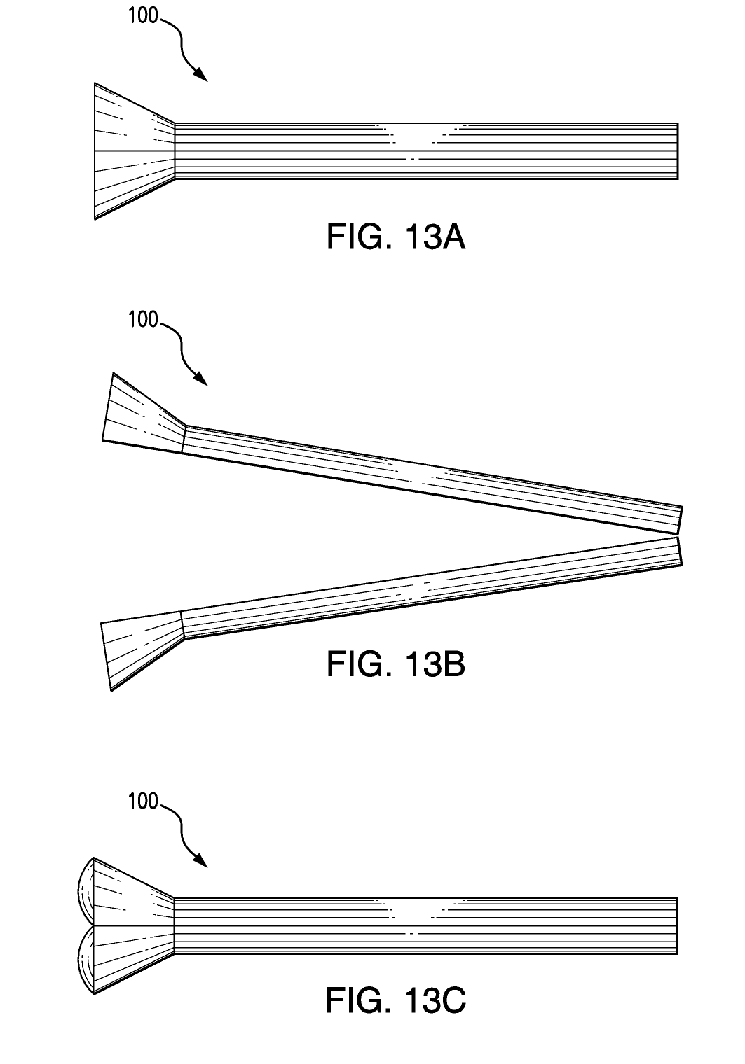

In accordance with another aspect of the disclosed subject matter, a method for inserting a balloon catheter into the vasculature of a patient is provided. Reference is made to FIGS. 8 to 11 for purpose of illustration and not limitation, which depict a preferred embodiment of the method in conjunction with an over-the-wire drug-coated balloon catheter. The method includes providing a catheter, an introducer sheath 100 and a guide catheter (or guide sheath) 230. The guide catheter 230 includes a tubular member with a lumen defined therein. The guide catheter can also include a hemostatic valve 240, such as when used for access to a blood vessel or cardiovascular system. Exemplary hemostatic valves include, for example, a rotating hemostatic valve, a bleedback control hemostatic valve, dual hemostatic valves, self-sealing hemostatic valves, or an integral plug-type hemostatic valve. An exemplary plug-type hemostatic valve is shown in FIGS. 12 to 14, for purpose of illustration and not limitation. In preparing the patient for insertion of the catheter, the physician makes an incision or percutaneous puncture and inserts a guide catheter into the blood vessel. The hemostatic valve of the guide catheter prevents back-bleeding from the proximal end of the guide catheter.

To avoid interaction between the hemostatic valve and the coating on the expandable member, the method further includes positioning the introducer sheath in a proximal end of the guide catheter as shown in FIGS. 8 and 9. The introducer sheath, which as noted above has an inner diameter sized to receive the catheter shaft and attached expandable member, protects the expandable member from making contact with the inner surfaces of the hemostatic valve and the guide catheter.

In accordance with this embodiment, the expandable member is first positioned within the introducer sheath as shown in FIG. 8. The introducer sheath containing the expandable member is then positioned into the guide catheter as shown in FIG. 9. Once the introducer sheath is in the guide catheter, the expandable member will be exposed to blood as it is pushed beyond the introducer sheath. Data suggests that therapeutic coatings are more resistant to abrasion and loss when the coatings are dry. Thus, positioning the expandable member within the introducer sheath prior to inserting the introducer sheath into the guide catheter is advantageous as it minimizes the distance that the expandable member will need to travel within the wet environment of the introducer sheath once in the guide catheter. Additionally, the expandable member can be slightly inflated within the introducer sheath. In this case the distal portion of the inflated expandable member will act as a plug, preventing or decreasing the exposure of the remainder of the expandable member to the patient's blood until it is pushed distally out of the introducer sheath. The expandable member can then be deflated to limit the surface area exposed to the wet environment of the patient's vasculature until it is positioned at the treatment site.

As also noted above, the introducer sheath can include an enlarged or flared portion at its proximal end. The enlarged cross section of the flared portion can be sized so that it is greater than the opening in the guide catheter thereby preventing the introducer sheath from completely passing into the guide catheter.

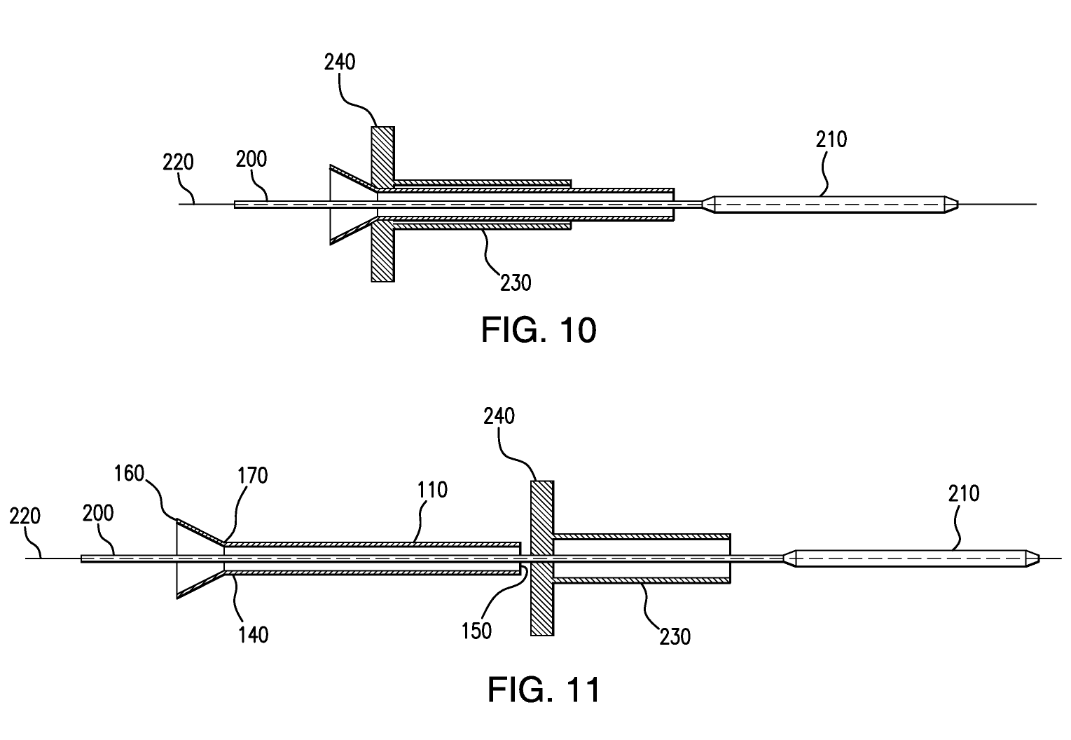

Reference is now made to FIGS. 10 and 11, for purposes of illustration and not limitation, depicting side views of an alternative method in accordance with the disclosed subject matter. After the introducer sheath and balloon catheter are positioned in the guide catheter, the balloon catheter is advanced distally beyond the distal end of the guide catheter into the vasculature of the patient as shown in FIG. 10. The introducer sheath can then be retracted proximally from the guide catheter as shown in FIG. 11. The introducer sheath can remain on the catheter shaft and/or guidewire. Additionally or alternatively, the introducer sheath can further include a breakable area of weakness as previously described. Furthermore, at least a portion of the introducer sheath can be brightly colored to ensure that it is noticed and removed prior to subsequent interventions.

While the disclosed subject matter is described herein in terms of certain preferred embodiments, those skilled in the art will recognize that various modifications and improvements can be made to the disclosed subject matter without departing from the scope thereof. Moreover, although individual features of one embodiment of the disclosed subject matter can be discussed herein or shown in the drawings of the one embodiment and not in other embodiments, it should be apparent that individual features of one embodiment can be combined with one or more features of another embodiment or features from a plurality of embodiments.

In addition to the specific embodiments claimed below, the disclosed subject matter is also directed to other embodiments having any other possible combination of the features disclosed above. As such, the particular features presented disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

1. An introducer sheath for use in inserting a balloon catheter into a patient comprising:
a tubular member having an inner diameter and an outer diameter, the tubular member further having a proximal end and a distal end with a length defined therebetween;
the inner diameter sized to receive a catheter shaft having an attached expandable member in a deflated condition;
the inner diameter being varied from a first inner diameter at the proximal end and a second inner diameter at the distal end, the first inner diameter being greater than the second inner diameter.

2. The introducer sheath of claim 1, wherein the proximal end of the introducer sheath includes a flared portion, the flared portion having a proximal end and a distal end, the proximal end of the flared portion having an enlarged cross section and the distal end of the flared portion having a cross section substantially similar to the cross section of the proximal end of the tubular member.

3. The introducer sheath of claim 1, further comprising a breakable area of weakness along at least a portion of the length of the introducer sheath from the proximal end to the distal end.

4. The introducer sheath of claim 3, wherein the breakable area of weakness comprises a perforation.

5. The introducer sheath of claim 2, further having an inner surface and an outer surface and a lubricious coating applied to at least one of the inner and outer surfaces of the introducer sheath.

6. The introducer sheath of claim 1, wherein the introducer sheath is formed of a material selected from the group consisting of: PEEK, HDPE, Kyner, FEP or PTFE.

7. A balloon catheter kit comprising:
a catheter including a tubular shaft having a proximal end a distal end and a length therebetween, the catheter further including an expandable member attached at the distal end of the tubular shaft, the expandable member having a length, an inner surface and an outer surface and further comprising a therapeutic coating applied to the outer surface of the expandable member;
an introducer sheath comprising:
a tubular member having an inner diameter and an outer diameter, the tubular member further having a proximal end and a distal end with a length defined therebetween,
the inner diameter sized to receive a catheter shaft having an attached expandable member in a deflated condition.

8. The kit of claim 7, wherein the length of the introducer sheath is sized to completely enclose the length of the expandable member when inserted into the introducer sheath.

9. The kit of claim 8, wherein the inner diameter is varied from a first inner diameter at the proximal end and a second inner diameter at the distal end, the first inner diameter being greater than the second inner diameter.

10. The kit of claim 7, wherein the proximal end of the introducer sheath includes a flared portion, the flared portion having a proximal end and a distal end, the proximal end of the flared portion having an enlarged cross section and the distal end of the flared portion having a cross section substantially similar to the cross section of the proximal end of the tubular member.

11. The kit of claim 7, wherein the introducer sheath further includes a breakable area of weakness along at least a portion of the length from the proximal end to the distal end of the introducer sheath.

12. The kit of claim 11, wherein the breakable area of weakness comprises a perforation.

13. The kit of claim 7, wherein the introducer sheath further has an inner surface and an outer surface and a lubricious coating applied to at least one of the inner and outer surfaces of the introducer sheath.

14. The kit of claim 7, wherein the introducer sheath is formed of a material selected from the group consisting of: PEEK, HDPE, Kyner, FEP and PTFE.

15. The kit of claim 7, wherein the therapeutic coating is selected from the group consisting of: Paclitaxel, Sirolimus, Ultravist and PVP.

16. The kit of claim 8, wherein the introducer sheath is disposed over the expandable member.

17. The kit of claim 7, further including a guide catheter, the guide catheter having an opening.

18. The kit of claim 17, wherein the enlarged cross section of the flared portion of the introducer sheath is greater than the size of the opening in the guide catheter, thereby preventing the introducer sheath from completely passing into the guide catheter.

19. A method of inserting a balloon catheter into the vasculature of a patient comprising:
providing a catheter having a shaft, the shaft having a proximal end, a distal end, and a length therebetween, the catheter further having an expandable member attached at its distal end;
providing a guide catheter comprising a tubular member with a lumen defined therein the guide catheter further having an opening;
providing an introducer sheath comprising:
a tubular member having an inner diameter and an outer diameter, the tubular member further having a proximal end and a distal end with a length defined therebetween,
the inner diameter sized to receive a catheter shaft having an attached expandable member in a deflated condition,
positioning the introducer sheath in a proximal end of the guide catheter; and
extending the expandable member through the introducer sheath and into the guide catheter.

20. The method of claim 19, wherein the inner diameter is varied from a first inner diameter at the proximal end and a second inner diameter at the distal end, the first inner diameter being greater than the second inner diameter.

21. The method of claim 19, wherein the proximal end of the introducer sheath includes a flared portion, the flared portion having a proximal end and a distal end, the proximal end of the flared portion having an enlarged cross section and the distal end of the flared portion having a cross section substantially similar to the cross section of the proximal end of the tubular member.

22. The method of claim 21, wherein the enlarged cross section of the flared portion of the introducer sheath is greater than the opening in the guide catheter, thereby preventing the introducer sheath from completely passing into the guide catheter.

23. The method of claim 19, wherein the introducer sheath further includes a breakable area of weakness along at least a portion of the length from the proximal end to the distal end of the introducer sheath, the method including breaking the breakable area of weakness to remove the introducer sheath from the catheter.

24. The method of claim 23, wherein the breakable area of weakness comprises a perforation.

25. The method of claim 19, wherein the introducer sheath further comprises an inner surface and an outer surface and wherein a lubricious coating is applied to at least one of the inner and outer surfaces of the introducer sheath.

26. The method of claim 19, wherein the introducer sheath is formed of a material selected from the group consisting of: PEEK, HDPE, Kyner, FEP and PTFE.

27. The method of claim 19, wherein the guide catheter includes a hemostatic valve and positioning the introducer sheath includes inserting the introducer sheath into the hemostatic valve.

28. The method of claim 27, wherein the introducer sheath is inserted into the hemostatic valve before extending the expandable member into the introducer sheath.

29. The method of claim 27, wherein the expandable member is extended into the introducer sheath before the introducer sheath is inserted into the hemostatic valve.

30. The method of claim 27, further comprising:
advancing the expandable member beyond a distal end of the guide catheter, and retracting the introducer sheath from the hemostatic valve.

31. The method of claim 19, wherein the catheter further has a guidewire lumen and further comprising:
providing a guidewire having a proximal end, a distal end, and a length therebetween;
inserting the distal end of the guidewire through the introducer sheath and into the vasculature of the patient after positioning the introducer sheath in the guide catheter;
and inserting the proximal end of the guidewire into the guidewire lumen of the catheter prior to extending the expandable member through the introducer sheath.
